SEQUENCE LISTING (4 sequences)

SEQ ID NO: 1 - protein, 31 aa, Homo sapiens
HAEGTFTSDVSSYLEGQAAKEFIAWLVKGRG

SEQ ID NO: 2 - protein, 31 aa, synthetic construct
HGEGTFTSDVSSYLEEQAAKEFIAWLVKGGG

SEQ ID NO: 3 - protein, 5 aa, synthetic construct
GGGGS

SEQ ID NO: 4 - protein, 275 aa, synthetic construct
HGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

(19) United States
(12) Patent Application Publication
(10) Pub. No.: US 2024/0318221 A1
(43) Pub. Date: Sep. 26, 2024

(54) METHODS OF PRODUCING FC-CONTAINING PROTEINS

(71) Applicant: ELI LILLY AND COMPANY, Indianapolis, IN (US)

(72) Inventors: Ciara DUNNE, Cork (IE); Brian Gerard KEARNS, Scotch Plains, NJ (US); Ashley Lynn RETTBERG, Hillsborough, NJ (US)

(21) Appl. No.: 18/612,180

(22) Filed: Mar. 21, 2024

Related U.S. Application Data
(60) Provisional application No. 63/491,758, filed on Mar. 23, 2023.

(51) Int. Cl. C12P 21/02; C07K 14/605; C12M 1/00; C12N 5/073

(52) U.S. Cl. CPC C12P 21/02; C07K 14/605; C12M 29/06; C12N 5/0604; C12N 2500/05

(57) ABSTRACT

Provided herein are improved methods for producing an Fc-containing protein. The methods generally involve culturing mammalian cells that produce the Fc-containing protein at a first pH setpoint for a first period of time followed by culturing the cell at a second pH setpoint that is higher than the first pH set point for a second period of time.

### REFERENCE TO A SEQUENCE LISTING

The present application is filed along with a Sequence Listing in ST.26 XML format. The Sequence Listing is provided as a filed titled “30171 US_Sequence Listing”, created on Mar. 19, 2024, and is 4,808 bytes in size. The Sequence Listing information in the ST.26 XML format is incorporated herein by reference in its entirety.

### BACKGROUND

Production of recombinant Fc-containing proteins for therapeutic use typically involves expression of the proteins in cultured mammalian cells. Cell culture conditions can affect the yield and/or quality of Fc-containing proteins produced by the cultured cells. In particular, sub-optimal cell culture conditions can result in an increase in the amount of protease clipping of Fc-containing proteins, such as dulaglutide, produced, which can impact yield and/or product quality.

Accordingly, there is a need for improved cell culture methods for producing recombinant Fc-containing proteins that maximize protein yield and quality.

### SUMMARY

The present disclosure provides improved methods for producing Fc-containing proteins. The methods generally involve culturing a mammalian cell that expresses the Fc-containing protein at a first pH setpoint for a first period of time followed by culturing the cell at a second pH setpoint that is higher than the first pH set point for a second period of time. The methods disclosed herein are particularly advantageous in that they can reduce protease clipping of Fc-containing proteins, such as dulaglutide.

In an aspect, provided herein is a method for producing dulaglutide, the method comprising the steps of a) culturing a mammalian cell that expresses the dulaglutide in a cell culture medium at a first pH setpoint for a first period of time; followed by b) culturing the mammalian cell in the cell culture medium at a second pH setpoint for a second period of time, wherein the second pH setpoint is higher than the first pH setpoint, such that the dulaglutide is produced by the mammalian cell.

In an embodiment, the first pH setpoint has a deadband of 0.01-0.10. In an embodiment, the first pH setpoint has a deadband of 0.07-0.10. In an embodiment, the first pH setpoint has a deadband of about 0.09.

In an embodiment, the second pH setpoint has a deadband of 0.01-0.10. In an embodiment, the second pH setpoint has a deadband of 0.03-0.06. In an embodiment, the second pH setpoint has a deadband of about 0.05.

In an embodiment, the deadband of the second pH setpoint is narrower than the deadband of the first pH setpoint. In an embodiment, the first pH setpoint has a deadband of about 0.09 and the second pH setpoint has a deadband of about 0.05.

In an embodiment, the second pH setpoint is 0.01-1.0 pH units higher than the first pH setpoint. In an embodiment, the first pH setpoint is 6.0-7.0. In an embodiment, the first pH setpoint is 6.5-6.9. In an embodiment, the first pH setpoint is 6.8-6.9. In an embodiment, the first pH setpoint is about 6.86.

In an embodiment, the second pH setpoint is 6.5-7.5. In an embodiment, the second pH setpoint is 6.9-7.5. In an embodiment, the second pH setpoint is 7.0-7.1. In an embodiment, the second pH setpoint is about 7.0.

In an embodiment, the first pH setpoint is about 6.86 with a deadband of about 0.09 and the second pH setpoint is about 7.0 with a deadband of about 0.05.

In an embodiment, the first period of time is longer than the second period of time. In an embodiment, the first period of time is 7-12 days. In an embodiment, the first period of time is 9-11 days. In an embodiment, the first period of time is about 10 days.

In an embodiment, the second period of time is at least 1 day. In an embodiment, the second period of time is 4-7 days. In an embodiment, the second period of time is 4-6 days. In an embodiment, the second period of time is about 5 days.

In an embodiment, the first period of time is about 10 days and the second period of time is about 5 days.

In an embodiment, the first pH setpoint is about 6.86 with a deadband of about 0.09 and the first period of time is 9-10 days; and the second pH setpoint is about 7.0 with a deadband of about 0.05 and the second period of time is 4-5 days.

In an embodiment, the method further comprises culturing the mammalian cell at a first temperature for 1-5 days followed by culturing the mammalian cell at a second temperature for 9-14 days. In an embodiment, the second temperature is lower than the first temperature.

In an embodiment, the first temperature is 34° C.-40° C. In an embodiment, the first temperature is about 36° C. In an embodiment, the second temperature is 30° C.-36° C. In an embodiment, the second temperature is about 33° C.

In an embodiment, the mammalian cell is cultured at the first temperature for 3-4 days. In an embodiment, the mammalian cell is cultured at the first temperature for about 3 days. In an embodiment, the mammalian cell is cultured at the second temperature for 11-12 days. In an embodiment, the mammalian cell is cultured at the second temperature for about 11 days.

In an embodiment, the mammalian cell is cultured in a bioreactor. In an embodiment, the mammalian cell is cultured in fed batch mode.

In an embodiment, the method further comprises adding a base source to the cell culture medium if the pH goes below the deadband or adding an acid source to the cell culture medium if the pH goes above the deadband during the first period of time. In an embodiment, the method further comprises adding a base source to the cell culture medium if the pH goes below the deadband or adding an acid source to the cell culture medium if the pH goes above the deadband during the second period of time. In an embodiment, the acid source is CO2. In an embodiment, the base source is a solution comprising NaOH.

In an embodiment, the mammalian cell culture medium is sparged with air during the first period of time. In an embodiment, the mammalian cell culture medium is sparged with air during the second period of time. In an embodiment, the mammalian cell culture medium is sparged with air during the first and second period of time.

In an embodiment, the mammalian cell is selected from the group consisting of a COS cell, a CHO cell, a BHK cell, an MDCK cell, a HEK293 cell, a HEK293T cell, a HeLa cell, an NSO cell, a PER.C6 cell, a VERO cell, a CRL7030 cell, an HsS78Bst cell, an NIH 3T3 cell, a HepG2 cell, an SP210 cell, an R1.1 cell, a B-W cell, an L-M cell, a BSC1 cell, a BSC40 cell, a YB/20 cell, and a BMT10 cell. In an embodiment, the mammalian cell is a CHO cell.

In an embodiment, one or more protease has reduced activity within the second pH deadband compared to the first pH deadband. In an embodiment, the one or more protease is in the cell or the cell culture medium. In an embodiment, one or more protease has reduced activity at the second pH setpoint compared to the first pH setpoint. In an embodiment, the one or more protease clips dulaglutide at the N-terminus. In an embodiment, the protease is cathepsin D. In an embodiment, less than about 4% of the dulaglutide produced is N-terminally clipped.

In an aspect, provided herein is dulaglutide produced by a method disclosed herein.

Additional embodiments of the present disclosure are described below:
1. A method for producing an Fc-containing protein, the method comprising the steps of.
a) culturing a mammalian cell that expresses the Fc-containing protein in a cell culture medium at a first pH setpoint for a first period of time; followed by
b) culturing the mammalian cell in the cell culture medium at a second pH setpoint for a second period of time, wherein the second pH setpoint is higher than the first pH setpoint, such that the Fc-containing protein is produced by the mammalian cell.
2. The method of embodiment 1, wherein the first pH setpoint has a deadband of 0.01-0.10.
3. The method of embodiment 1 or 2, wherein the first pH setpoint has a deadband of 0.07-0.10.
4. The method of any one of the preceding embodiments, wherein the first pH setpoint has a deadband of about 0.09.
5. The method of any one of the preceding embodiments, wherein the second pH setpoint has a deadband of 0.01-0.10.
6. The method of any one of the preceding embodiments, wherein the second pH setpoint has a deadband of 0.03-0.06.
7. The method of any one of the preceding embodiments, wherein the second pH setpoint has a deadband of about 0.05.
8. The method of any one of the preceding embodiments, wherein the deadband of the second pH setpoint is narrower than the deadband of the first pH setpoint.
9. The method of any one of the preceding embodiments, wherein the first pH setpoint has a deadband of about 0.09 and the second pH setpoint has a deadband of about 0.05.
10. The method of any one of the preceding embodiments, wherein the second pH setpoint is 0.01-1.0 pH units higher than the first pH setpoint.
11. The method of any one of the preceding embodiments, wherein the first pH setpoint is 6.0-7.0.
12. The method of any one of the preceding embodiments, wherein the first pH setpoint is 6.5-6.9.
13. The method of any one of the preceding embodiments, wherein the first pH setpoint is 6.8-6.9.
14. The method of any one of the preceding embodiments, wherein the first pH setpoint is about 6.86.
15. The method of any one of the preceding embodiments, wherein the second pH setpoint is 6.5-7.5.
16. The method of any one of the preceding embodiments, wherein the second pH setpoint is 6.9-7.5.
17. The method of any one of the preceding embodiments, wherein the second pH setpoint is 7.0-7.1.
18. The method of any one of the preceding embodiments, wherein the second pH setpoint is about 7.0.
19. The method of any one of the preceding embodiments, wherein the first pH setpoint is about 6.86 with a deadband of about 0.09 and the second pH setpoint is about 7.0 with a deadband of about 0.05.
20. The method of any one of the preceding embodiments, wherein the first period of time is longer than the second period of time.
21. The method of any one of the preceding embodiments, wherein the first period of time is 7-12 days.
22. The method of any one of the preceding embodiments, wherein the first period of time is 9-11 days.
23. The method of any one of the preceding embodiments, wherein the first period of time is about 10 days.
24. The method of any one of the preceding embodiments, wherein the second period of time is at least 1 day.
25. The method of any one of the preceding embodiments, wherein the second period of time is 4-7 days.
26. The method of any one of the preceding embodiments, wherein the second period of time is 4-6 days.
27. The method of any one of the preceding embodiments, wherein the second period of time is about 5 days.
28. The method of any one of the preceding embodiments, wherein the first period of time is about 10 days and the second period of time is about 5 days.
29. The method of any one of the preceding embodiments, wherein:
a) the first pH setpoint is about 6.86 with a deadband of about 0.09 and the first period of time is 9-10 days; and
b) the second pH setpoint is about 7.0 with a deadband of about 0.05 and the second period of time is 4-5 days.
30. The method of any one of embodiments 1-22, further comprising culturing the mammalian cell at a first temperature for the first 1-5 days of the first period of time followed by culturing the mammalian cell at a second temperature for 9-14 days.
31. The method of embodiment 30, wherein the second temperature is lower than the first temperature.
32. The method of embodiment 30 or 31, wherein the first temperature is 34° C.-40° C.
33. The method of any one of embodiments 30-32, wherein the first temperature is about 36° C.
34. The method of any one of embodiments 30-33, wherein the second temperature is 30° C.-36° C.
35. The method of any one of embodiments 30-34, wherein the second temperature is about 33° C.
36. The method of any one of embodiments 30-35, wherein the mammalian cell is cultured at the first temperature for 3-4 days.
37. The method of any one of embodiments 30-36, wherein the mammalian cell is cultured at the first temperature for about 3 days.
38. The method of any one of embodiments 30-37, wherein the mammalian cell is cultured at the second temperature for 11-12 days.
39. The method of any one of embodiments 30-38, wherein the mammalian cell is cultured at the second temperature for about 11 days.
40. The method of any one of the preceding embodiments, wherein the mammalian cell is cultured in a bioreactor.
41. The method of embodiment 40, wherein the mammalian cell is cultured in fed batch mode.
42. The method of any one of the preceding embodiments, wherein the mammalian cell is selected from the group consisting of a COS cell, a CHO cell, a BHK cell, an MDCK cell, a HEK293 cell, a HEK293T cell, a HeLa cell, an NSO cell, a PER.C6 cell, a VERO cell, a CRL7030 cell, an HsS78Bst cell, an NIH 3T3 cell, a HepG2 cell, an SP210 cell, an R1.1 cell, a B-W cell, an L-M cell, a BSC1 cell, a BSC40 cell, a YB/20 cell, and a BMT10 cell.
43. The method of any one of the preceding embodiments, wherein the mammalian cell is a CHO cell.
44. The method of any one of the preceding embodiments, further comprising adding a base source to the cell culture medium if the pH goes below the deadband or adding an acid source to the cell culture medium if the pH goes above the deadband during the first period of time.
45. The method of any one of the preceding embodiments, further comprising adding a base source to the cell culture medium if the pH goes below the deadband or adding an acid source to the cell culture medium if the pH goes above the deadband during the second period of time.
46. The method of embodiment 44 or 45, wherein the acid source is CO₂. 47. The method of embodiment 44 or 45, wherein the base source is a solution comprising NaOH.
48. The method of any one of the preceding embodiments, wherein the cell culture medium is sparged with air during the first and/or second period of time.
49. The method of any one of the preceding embodiments, wherein one or more protease has reduced activity within the second pH deadband compared to the first pH deadband.
50. The method of any one of the preceding embodiments, wherein one or more protease has reduced activity at the second pH setpoint compared to the first pH setpoint.
51. The method of embodiment 49 or 50, wherein the one or more protease clips the Fc-containing protein at the N-terminus, optionally wherein the protease is cathepsin D.
52. The method of any one of the preceding embodiments, wherein the mammalian cell culture comprises less than about 4% of a clipped form of the Fc-containing protein at the end of the second period of time.
53. The method of any one of the preceding embodiments, wherein the Fc-containing protein comprises a glucagon-like peptide 1 (GLP-1) analog comprising one or more modifications compared to a wild type GLP-1 amino acid sequence (SEQ ID NO: 1).
54. The method of any one of the preceding embodiments, wherein the Fc-containing protein comprises a GLP-1 analog comprising the amino acid sequence of SEQ ID NO: 2.
55. The method of any one of the preceding embodiments, wherein the Fc-containing protein comprises a peptide linker.
56. The method of embodiment 55, wherein the peptide linker comprises 1-10 G₄S units (SEQ ID NO: 3).
57. The method of embodiment 55 or 56, wherein the peptide linker comprises the amino acid sequence set forth in SEQ ID NO: 3.
58. The method of any one of the preceding embodiments, wherein the Fc-containing protein comprises:
a) a GLP-1 analog comprising the amino acid sequence of SEQ ID NO: 2;
b) a peptide linker comprising the amino acid sequence of SEQ ID NO: 3; and
c) an Fc portion of an immunoglobulin.
59. The method of embodiment 58, wherein the N-terminal residue of the peptide linker is directly fused to the C-terminal residue of the GLP-1 analog, and the C-terminal residue of the peptide linker is directly fused to the N-terminal residue of the Fc portion.
60. The method of any one of the preceding embodiments, wherein the Fc-containing protein comprises the amino acid sequence of SEQ ID NO: 4.
61. The method of any one of the preceding embodiments, wherein the wherein the Fc-containing protein is a homodimer comprising two identical amino acid chains each comprising the amino acid sequence of SEQ ID NO: 4.
62. The method of any one of the preceding embodiments, wherein the Fc-containing protein is dulaglutide.
63. A method for producing dulaglutide in a CHO cell, the method comprising the steps of:
a) culturing the CHO cell in a cell culture medium maintained at a first pH setpoint of about 6.68 having a deadband of about 0.09 for about 10 days; followed by
b) culturing the CHO cell in the cell culture medium maintained at a second pH setpoint of about 7.0 having a deadband of about 0.05 for about 5 days; and wherein the culturing is initiated at a temperature of about 36° C. and then decreased to about 33° C. after about 3 days; such that the dulaglutide is produced by the CHO cell.
64. The method of embodiment 63 wherein the CHO cell is cultured in a bioreactor in fed batch mode.
65. The method of either of embodiments 63-64 wherein NaOH is added to the cell culture medium if the pH goes below the deadband of either the first or second pH setpoint, and CO₂ is added to the cell culture medium if the pH goes above the deadband of either the first or second pH setpoint.
66. The method of any one of embodiments 63-65 wherein the cell culture medium is sparged with air one or more times.
67. The method of any one of embodiments 63-67 wherein less than about 4% of the dulaglutide produced by the CHO cell is N-terminally clipped.
64. An Fc-containing protein produced by the method of any one of the preceding embodiments.

### BRIEF DESCRIPTION OF DRAWINGS

FIG. 1 is a graph showing pCO₂ levels in the production bioreactor over a 14 day culture for two different runs at pH 6.80±0.03 or two different runs at pH 6.86±0.09.

FIG. 2 is a plot showing day 14 pH levels and des H/HG levels in 3 L and 5 L production bioreactors.

### DETAILED DESCRIPTION

The present disclosure provides improved methods for producing Fc-containing proteins. The methods generally involve culturing a mammalian cell that expresses the Fc-containing protein at a first pH setpoint for a first period of time followed by culturing the cell at a second pH setpoint that is higher than the first pH set point for a second period of time. The methods disclosed herein are particularly advantageous in that they can reduce protease clipping of Fc-containing proteins.

### I. Definitions

As used herein, the term “pH setpoint” refers to a desired pH value for a cell culture medium. In certain embodiments, a pH setpoint for a cell culture medium is actively maintained (e.g., by the addition of acid or base to the cell culture medium) in response to changes in pH that falls outside of a specified deadband.

As used herein, the term “deadband” refers to the range of pH values above or below a pH setpoint within which the pH of a cell culture medium is permitted to vary without intervention. For example, the pH of a cell culture medium with a pH setpoint of 7.00 and a deadband of 0.05 can vary from pH 6.95 to 7.05. In general, the pH setpoint and deadband are expressed as “setpoint±deadband,” e.g., 7.00±0.05.

As used herein, the term “pH shift” or “pH setpoint shift” refers to a change in the pH of a cell culture medium caused by actively altering the pH setpoint to a lower or higher value.

As used herein, the term “Fc-containing protein” refers to a protein comprising an Fc region. In an embodiment, the Fc-containing protein comprises a variant Fc region comprising one or more amino acid substitutions, additions, and/or deletions relative to a naturally occurring Fc region. In an embodiment, the Fc-containing protein is an antibody. In an embodiment, the Fc-containing protein is not an antibody.

As used herein, the term “antibody” includes full-length antibodies, antigen-binding fragments of full-length antibodies, and molecules comprising antibody CDRs, VH regions, and/or VL regions. Examples of antibodies include, without limitation, monoclonal antibodies, recombinantly produced antibodies, monospecific antibodies, multispecific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, antibody-drug conjugates, single domain antibodies, monovalent antibodies, single chain antibodies or single-chain Fvs (scFv), camelized antibodies, affibodies, Fab fragments, F(ab′)2 fragments, disulfide-linked Fvs (sdFv), anti-idiotypic (anti-Id) antibodies (including, e.g., anti-anti-Id antibodies), and antigen-binding fragments of any of the above.

As used herein, the term “about,” when in reference to a value or parameter herein, includes a variability of ±5% of the value or parameter. For example, when referring to a pH value, “about” refers to a range that includes the value 5% below the referenced value, and the value 5% above the referenced value. Thus, a pH of about 10 refers to a pH that encompasses a pH of 9.5 to a pH of 10.5, inclusive.

### II. Methods of Culturing Mammalian Cells

When producing an Fc-containing protein, optimizing the pH conditions of a mammalian cell culture in a production bioreactor is important to maintain the product quality of the Fc-containing protein. Disclosed herein are methods of producing an Fc-containing protein comprising culturing mammalian cells that express the Fc-containing protein at a first pH setpoint followed by a shift to a second pH setpoint that is higher than the first pH setpoint. The methods disclosed herein are particularly advantageous in that they can reduce the protease clipping of the Fc-containing protein in the mammalian cell culture thereby increasing the amount of intact Fc-containing protein.

In an aspect, the methods disclosed herein generally comprise the steps of: a) culturing a mammalian cell that expresses the Fc-containing protein in a cell culture medium at a first pH setpoint for a first period of time; followed by b) culturing the mammalian cell in the cell culture medium at a second pH setpoint for a second period of time, wherein the second pH setpoint is higher than the first pH setpoint, such that the Fc-containing protein is produced by the mammalian cell.

In an aspect, the methods disclosed herein generally comprise the steps of: a) culturing a mammalian cell that expresses the dulaglutide in a cell culture medium at a first pH setpoint for a first period of time; followed by b) culturing the mammalian cell in the cell culture medium at a second pH setpoint for a second period of time, wherein the second pH setpoint is higher than the first pH setpoint, such that dulaglutide is produced by the mammalian cell.

Exemplary cell culture methods and conditions suitable for use in the foregoing methods are described in detail below.

In an embodiment, the first pH setpoint has a deadband of 0.01-0.10. In an embodiment, the first pH setpoint has a deadband of 0.07-0.12. In an embodiment, the first pH setpoint has a deadband of 0.07-0.10. In an embodiment, the first pH setpoint has a deadband of about 0.01, about 0.02, about 0.03, about 0.04, about 0.05, about 0.06, about 0.07, about 0.08, about 0.09, about 0.1, about 0.11, about 0.12, about 0.13, about 0.14, or about 0.15. In an embodiment, the first pH setpoint has a deadband of about 0.09.

In an embodiment, the second pH setpoint has a deadband of 0.01-0.10. In an embodiment, the second pH setpoint has a deadband of 0.03-0.08. In an embodiment, the second pH setpoint has a deadband of 0.03-0.06. In an embodiment, the second pH setpoint has a deadband of about 0.01, about 0.02, about 0.03, about 0.04, about 0.05, about 0.06, about 0.07, about 0.08, about 0.09, about 0.1, about 0.11, about 0.12, about 0.13, about 0.14, or about 0.15. In an embodiment, the second pH setpoint has a deadband of about 0.05.

In an embodiment, the deadband of the second pH setpoint is narrower than the deadband of the first pH setpoint. In an embodiment, the first pH setpoint has a deadband of about 0.09 and the second pH setpoint has a deadband of about 0.05.

In an embodiment, the second pH setpoint is 0.01-1.0 pH units higher than the first pH setpoint. In an embodiment, the second pH setpoint is about 0.01, about 0.05, about 0.1, about 0.15, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 0.95, or about 1.0 pH units higher than the first pH setpoint. In an embodiment, the second pH setpoint is 0.1-0.2 pH units higher than the first pH setpoint. In an embodiment, the second pH setpoint is about 0.1, about 0.11, about 0.12, about 0.13, about 0.14, about 0.15, about 0.16, about 0.17, about 0.18, about 0.19, or about 0.2 pH units higher than the first pH setpoint.

In an embodiment, the first pH setpoint is 6.0-7.0. In an embodiment, the first pH setpoint is 6.5-6.9. In an embodiment, the first pH setpoint is 6.8-6.9. In an embodiment, the first pH setpoint is 6.85-7.0. In an embodiment, the first pH setpoint is about 6.8, about 6.81, about 6.82, about 6.83, about 6.84, about 6.85, about 6.86, about 6.87, about 6.88, about 6.89, or about 6.9. In an embodiment, the first pH setpoint is about 6.86.

In an embodiment, the first pH setpoint is about 6.8, about 6.81, about 6.82, about 6.83, about 6.84, about 6.85, about 6.86, about 6.87, about 6.88, about 6.89, or about 6.9 with a deadband of about 0.01, about 0.02, about 0.03, about 0.04, about 0.05, about 0.06, about 0.07, about 0.08, about 0.09, about 0.1, about 0.11, about 0.12, about 0.13, about 0.14, or about 0.15. In an embodiment, the first pH setpoint is about 6.86 with a deadband of about 0.01, about 0.02, about 0.03, about 0.04, about 0.05, about 0.06, about 0.07, about 0.08, about 0.09, about 0.1, about 0.11, about 0.12, about 0.13, about 0.14, or about 0.15.

In an embodiment, the first pH setpoint is about 6.8, about 6.81, about 6.82, about 6.83, about 6.84, about 6.85, about 6.86, about 6.87, about 6.88, about 6.89, or about 6.9 with a deadband of about 0.01.

In an embodiment, the first pH setpoint is about 6.8, about 6.81, about 6.82, about 6.83, about 6.84, about 6.85, about 6.86, about 6.87, about 6.88, about 6.89, or about 6.9 with a deadband of about 0.02.

In an embodiment, the first pH setpoint is about 6.8, about 6.81, about 6.82, about 6.83, about 6.84, about 6.85, about 6.86, about 6.87, about 6.88, about 6.89, or about 6.9 with a deadband of about 0.03.

In an embodiment, the first pH setpoint is about 6.8, about 6.81, about 6.82, about 6.83, about 6.84, about 6.85, about 6.86, about 6.87, about 6.88, about 6.89, or about 6.9 with a deadband of about 0.04.

In an embodiment, the first pH setpoint is about 6.8, about 6.81, about 6.82, about 6.83, about 6.84, about 6.85, about 6.86, about 6.87, about 6.88, about 6.89, or about 6.9 with a deadband of about 0.05.

In an embodiment, the first pH setpoint is about 6.8, about 6.81, about 6.82, about 6.83, about 6.84, about 6.85, about 6.86, about 6.87, about 6.88, about 6.89, or about 6.9 with a deadband of about 0.06.

In an embodiment, the first pH setpoint is about 6.8, about 6.81, about 6.82, about 6.83, about 6.84, about 6.85, about 6.86, about 6.87, about 6.88, about 6.89, or about 6.9 with a deadband of about 0.07.

In an embodiment, the first pH setpoint is about 6.8, about 6.81, about 6.82, about 6.83, about 6.84, about 6.85, about 6.86, about 6.87, about 6.88, about 6.89, or about 6.9 with a deadband of about 0.08.

In an embodiment, the first pH setpoint is about 6.8, about 6.81, about 6.82, about 6.83, about 6.84, about 6.85, about 6.86, about 6.87, about 6.88, about 6.89, or about 6.9 with a deadband of about 0.09.

In an embodiment, the first pH setpoint is about 6.8, about 6.81, about 6.82, about 6.83, about 6.84, about 6.85, about 6.86, about 6.87, about 6.88, about 6.89, or about 6.9 with a deadband of about 0.1.

In an embodiment, the first pH setpoint is about 6.8, about 6.81, about 6.82, about 6.83, about 6.84, about 6.85, about 6.86, about 6.87, about 6.88, about 6.89, or about 6.9 with a deadband of about 0.11.

In an embodiment, the first pH setpoint is about 6.8, about 6.81, about 6.82, about 6.83, about 6.84, about 6.85, about 6.86, about 6.87, about 6.88, about 6.89, or about 6.9 with a deadband of about 0.12.

In an embodiment, the first pH setpoint is about 6.8, about 6.81, about 6.82, about 6.83, about 6.84, about 6.85, about 6.86, about 6.87, about 6.88, about 6.89, or about 6.9 with a deadband of about 0.13.

In an embodiment, the first pH setpoint is about 6.8, about 6.81, about 6.82, about 6.83, about 6.84, about 6.85, about 6.86, about 6.87, about 6.88, about 6.89, or about 6.9 with a deadband of about 0.14.

In an embodiment, the first pH setpoint is about 6.8, about 6.81, about 6.82, about 6.83, about 6.84, about 6.85, about 6.86, about 6.87, about 6.88, about 6.89, or about 6.9 with a deadband of about 0.15.

In an embodiment, the second pH setpoint is 6.5-7.5. In an embodiment, the second pH setpoint is 6.9-7.5. In an embodiment, the second pH setpoint is 6.9-7.1. In an embodiment, the second pH setpoint is 7.0-7.1. In an embodiment, the second pH setpoint is about 6.9, about 6.9, about 6.92, about 6.93, about 6.94, about 6.95, about 6.96, about 6.97, about 6.98, about 6.99, about 7.0, about 7.01, about 7.02, about 7.03, about 7.04, about 7.05, about 7.06, about 7.07, about 7.08, about 7.09, or about 7.1. In an embodiment, the second pH setpoint is about 7.0.

In an embodiment, the second pH setpoint is about 6.9, about 6.9, about 6.92, about 6.93, about 6.94, about 6.95, about 6.96, about 6.97, about 6.98, about 6.99, about 7.0, about 7.01, about 7.02, about 7.03, about 7.04, about 7.05, about 7.06, about 7.07, about 7.08, about 7.09, or about 7.1 with a deadband of about 0.01, about 0.02, about 0.03, about 0.04, about 0.05, about 0.06, about 0.07, about 0.08, about 0.09, about 0.1, about 0.11, about 0.12, about 0.13, about 0.14, or about 0.15. In an embodiment, the second pH setpoint is about 7.0 with a deadband of about 0.01, about 0.02, about 0.03, about 0.04, about 0.05, about 0.06, about 0.07, about 0.08, about 0.09, about 0.1, about 0.11, about 0.12, about 0.13, about 0.14, or about 0.15.

In an embodiment, the second pH setpoint is about 6.9, about 6.9, about 6.92, about 6.93, about 6.94, about 6.95, about 6.96, about 6.97, about 6.98, about 6.99, about 7.0, about 7.01, about 7.02, about 7.03, about 7.04, about 7.05, about 7.06, about 7.07, about 7.08, about 7.09, or about 7.1 with a deadband of about 0.01.

In an embodiment, the second pH setpoint is about 6.9, about 6.9, about 6.92, about 6.93, about 6.94, about 6.95, about 6.96, about 6.97, about 6.98, about 6.99, about 7.0, about 7.01, about 7.02, about 7.03, about 7.04, about 7.05, about 7.06, about 7.07, about 7.08, about 7.09, or about 7.1 with a deadband of about 0.02.

In an embodiment, the second pH setpoint is about 6.9, about 6.9, about 6.92, about 6.93, about 6.94, about 6.95, about 6.96, about 6.97, about 6.98, about 6.99, about 7.0, about 7.01, about 7.02, about 7.03, about 7.04, about 7.05, about 7.06, about 7.07, about 7.08, about 7.09, or about 7.1 with a deadband of about 0.03.

In an embodiment, the second pH setpoint is about 6.9, about 6.9, about 6.92, about 6.93, about 6.94, about 6.95, about 6.96, about 6.97, about 6.98, about 6.99, about 7.0, about 7.01, about 7.02, about 7.03, about 7.04, about 7.05, about 7.06, about 7.07, about 7.08, about 7.09, or about 7.1 with a deadband of about 0.04.

In an embodiment, the second pH setpoint is about 6.9, about 6.9, about 6.92, about 6.93, about 6.94, about 6.95, about 6.96, about 6.97, about 6.98, about 6.99, about 7.0, about 7.01, about 7.02, about 7.03, about 7.04, about 7.05, about 7.06, about 7.07, about 7.08, about 7.09, or about 7.1 with a deadband of about 0.05.

In an embodiment, the second pH setpoint is about 6.9, about 6.9, about 6.92, about 6.93, about 6.94, about 6.95, about 6.96, about 6.97, about 6.98, about 6.99, about 7.0, about 7.01, about 7.02, about 7.03, about 7.04, about 7.05, about 7.06, about 7.07, about 7.08, about 7.09, or about 7.1 with a deadband of about 0.06.

In an embodiment, the second pH setpoint is about 6.9, about 6.9, about 6.92, about 6.93, about 6.94, about 6.95, about 6.96, about 6.97, about 6.98, about 6.99, about 7.0, about 7.01, about 7.02, about 7.03, about 7.04, about 7.05, about 7.06, about 7.07, about 7.08, about 7.09, or about 7.1 with a deadband of about 0.07.

In an embodiment, the second pH setpoint is about 6.9, about 6.9, about 6.92, about 6.93, about 6.94, about 6.95, about 6.96, about 6.97, about 6.98, about 6.99, about 7.0, about 7.01, about 7.02, about 7.03, about 7.04, about 7.05, about 7.06, about 7.07, about 7.08, about 7.09, or about 7.1 with a deadband of about 0.08.

In an embodiment, the second pH setpoint is about 6.9, about 6.9, about 6.92, about 6.93, about 6.94, about 6.95, about 6.96, about 6.97, about 6.98, about 6.99, about 7.0, about 7.01, about 7.02, about 7.03, about 7.04, about 7.05, about 7.06, about 7.07, about 7.08, about 7.09, or about 7.1 with a deadband of about 0.09.

In an embodiment, the second pH setpoint is about 6.9, about 6.9, about 6.92, about 6.93, about 6.94, about 6.95, about 6.96, about 6.97, about 6.98, about 6.99, about 7.0, about 7.01, about 7.02, about 7.03, about 7.04, about 7.05, about 7.06, about 7.07, about 7.08, about 7.09, or about 7.1 with a deadband of about 0.1.

In an embodiment, the second pH setpoint is about 6.9, about 6.9, about 6.92, about 6.93, about 6.94, about 6.95, about 6.96, about 6.97, about 6.98, about 6.99, about 7.0, about 7.01, about 7.02, about 7.03, about 7.04, about 7.05, about 7.06, about 7.07, about 7.08, about 7.09, or about 7.1 with a deadband of about 0.11.

In an embodiment, the second pH setpoint is about 6.9, about 6.9, about 6.92, about 6.93, about 6.94, about 6.95, about 6.96, about 6.97, about 6.98, about 6.99, about 7.0, about 7.01, about 7.02, about 7.03, about 7.04, about 7.05, about 7.06, about 7.07, about 7.08, about 7.09, or about 7.1 with a deadband of about 0.12.

In an embodiment, the second pH setpoint is about 6.9, about 6.9, about 6.92, about 6.93, about 6.94, about 6.95, about 6.96, about 6.97, about 6.98, about 6.99, about 7.0, about 7.01, about 7.02, about 7.03, about 7.04, about 7.05, about 7.06, about 7.07, about 7.08, about 7.09, or about 7.1 with a deadband of about 0.13.

In an embodiment, the second pH setpoint is about 6.9, about 6.9, about 6.92, about 6.93, about 6.94, about 6.95, about 6.96, about 6.97, about 6.98, about 6.99, about 7.0, about 7.01, about 7.02, about 7.03, about 7.04, about 7.05, about 7.06, about 7.07, about 7.08, about 7.09, or about 7.1 with a deadband of about 0.14.

In an embodiment, the second pH setpoint is about 6.9, about 6.9, about 6.92, about 6.93, about 6.94, about 6.95, about 6.96, about 6.97, about 6.98, about 6.99, about 7.0, about 7.01, about 7.02, about 7.03, about 7.04, about 7.05, about 7.06, about 7.07, about 7.08, about 7.09, or about 7.1 with a deadband of about 0.15.

In an embodiment, the first pH setpoint is 6.0-7.0 and the second pH setpoint is about 7.0. In an embodiment, the first pH setpoint is 6.5-6.9 and the second pH setpoint is about 7.0. In an embodiment, the first pH setpoint is 6.8-6.9 and the second pH setpoint is about 7.0. In an embodiment, the first pH setpoint is 6.85-7.0 and the second pH setpoint is about 7.0. In an embodiment, the first pH setpoint is about 6.8, about 6.81, about 6.82, about 6.83, about 6.84, about 6.85, about 6.86, about 6.87, about 6.88, about 6.89, or about 6.9 and the second pH setpoint is about 7.0.

In an embodiment, the first pH setpoint is about 6.86 and the second pH setpoint is 6.5-7.5. In an embodiment, the first pH setpoint is about 6.86 and the second pH setpoint is 6.9-7.5. In an embodiment, the first pH setpoint is about 6.86 and the second pH setpoint is 6.9-7.1. In an embodiment, the first pH setpoint is about 6.86 and the second pH setpoint is 7.0-7.1. In an embodiment, the first pH setpoint is about 6.86 and the second pH setpoint is about 6.9, about 6.9, about 6.92, about 6.93, about 6.94, about 6.95, about 6.96, about 6.97, about 6.98, about 6.99, about 7.0, about 7.01, about 7.02, about 7.03, about 7.04, about 7.05, about 7.06, about 7.07, about 7.08, about 7.09, or about 7.1. In an embodiment, the first pH setpoint is about 6.86 and the second pH setpoint is about 7.0.

In an embodiment, the first pH setpoint is about 6.86 with a deadband of about 0.09 and the second pH setpoint is about 7.0 with a deadband of about 0.05.

In an embodiment, the first period of time is longer than the second period of time. In an embodiment, the first period of time is 7-12 days. In an embodiment, the first period of time is 9-11 days. In an embodiment, the first period of time is about 10 days. In an embodiment, the first period of time is about 7 days, about 8 days, about 9 days, about 10 days, about 11 days, or about 12 days. In an embodiment, the first period of time and the second period of time add up to about 14 days.

In an embodiment, the second period of time is at least 1 day. In an embodiment, the second period of time is 4-7 days. In an embodiment, the second period of time is 4-6 days. In an embodiment, the second period of time is about 4 days, about 5 days, about 6 days, or about 7 days. In an embodiment, the second period of time is about 5 days.

In an embodiment, the first period of time is about 8 days and the second period of time is at least 1 day. In an embodiment, the first period of time is about 8 days and the second period of time is 4-7 days. In an embodiment, the first period of time is about 8 days and the second period of time is 4-6 days. In an embodiment, the first period of time is about 8 days and the second period of time is about 4 days, about 5 days, about 6 days, or about 7 days.

In an embodiment, the first period of time is about 9 days and the second period of time is at least 1 day. In an embodiment, the first period of time is about 9 days and the second period of time is 4-7 days. In an embodiment, the first period of time is about 9 days and the second period of time is 4-6 days. In an embodiment, the first period of time is about 9 days and the second period of time is about 4 days, about 5 days, about 6 days, or about 7 days.

In an embodiment, the first period of time is about 10 days and the second period of time is at least 1 day. In an embodiment, the first period of time is about 10 days and the second period of time is 4-7 days. In an embodiment, the first period of time is about 10 days and the second period of time is 4-6 days. In an embodiment, the first period of time is about 10 days and the second period of time is about 4 days, about 5 days, about 6 days, or about 7 days.

In an embodiment, the first period of time is about 11 days and the second period of time is at least 1 day. In an embodiment, the first period of time is about 11 days and the second period of time is 4-7 days. In an embodiment, the first period of time is about 11 days and the second period of time is 4-6 days. In an embodiment, the first period of time is about 11 days and the second period of time is about 4 days, about 5 days, about 6 days, or about 7 days.

In an embodiment, the first period of time is about 11 days and the second period of time is about 4 days. In an embodiment, the first period of time is about 11 days and the second period of time is about 3 days. In an embodiment, the first period of time is about 10 days and the second period of time is about 5 days. In an embodiment, the first period of time is about 10 days and the second period of time is about 4 days. In an embodiment, the first period of time is about 9 days and the second period of time is about 6 days. In an embodiment, the first period of time is about 9 days and the second period of time is about 5 days.

In an embodiment, the first pH setpoint is 6.8-6.9 with a deadband of 0.08-0.1 and the first period of time is 9-10 days; and the second pH setpoint is 7.0-7.1 with a deadband of 0.04-0.06 and the second period of time is 4-5 days.

In an embodiment, the first pH setpoint is 6.8-6.9 with a deadband of 0.08-0.1 and the first period of time is about 9 days; and the second pH setpoint is 7.0-7.1 with a deadband of 0.04-0.06 and the second period of time is about 6 days.

In an embodiment, the first pH setpoint is 6.8-6.9 with a deadband of 0.08-0.1 and the first period of time is about 9 days; and the second pH setpoint is 7.0-7.1 with a deadband of 0.04-0.06 and the second period of time is about 5 days.

In an embodiment, the first pH setpoint is 6.8-6.9 with a deadband of 0.08-0.1 and the first period of time is about 10 days; and the second pH setpoint is 7.0-7.1 with a deadband of 0.04-0.06 and the second period of time is about 5 days.

In an embodiment, the first pH setpoint is 6.8-6.9 with a deadband of 0.08-0.1 and the first period of time is about 10 days; and the second pH setpoint is 7.0-7.1 with a deadband of 0.04-0.06 and the second period of time is about 4 days.

In an embodiment, the first pH setpoint is 6.8-6.9 with a deadband of 0.08-0.1 and the first period of time is about 11 days; and the second pH setpoint is 7.0-7.1 with a deadband of 0.04-0.06 and the second period of time is about 4 days.

In an embodiment, the first pH setpoint is 6.8-6.9 with a deadband of 0.08-0.1 and the first period of time is about 11 days; and the second pH setpoint is 7.0-7.1 with a deadband of 0.04-0.06 and the second period of time is about 3 days.

In an embodiment, the first pH setpoint is about 6.86 with a deadband of about 0.09 and the first period of time is 9-11 days; and the second pH setpoint is about 7.0 with a deadband of about 0.05 and the second period of time is 4-6 days.

In an embodiment, the first pH setpoint is about 6.86 with a deadband of about 0.09 and the first period of time is 9-11 days; and the second pH setpoint is about 7.0 with a deadband of about 0.05 and the second period of time is about 4 days.

In an embodiment, the first pH setpoint is about 6.86 with a deadband of about 0.09 and the first period of time is 9-11 days; and the second pH setpoint is about 7.0 with a deadband of about 0.05 and the second period of time is about 5 days.

In an embodiment, the first pH setpoint is about 6.86 with a deadband of about 0.09 and the first period of time is 9-11 days; and the second pH setpoint is about 7.0 with a deadband of about 0.05 and the second period of time is about 6 days.

In an embodiment, the method further comprises culturing the mammalian cell at a first temperature for 1-5 days followed by culturing the mammalian cell at a second temperature for 9-14 days. In an embodiment, the method further comprises culturing the mammalian cell at a first temperature for about 1 day followed by culturing the mammalian cell at a second temperature for about 9, about 10, about 11, about 12, about 13, or about 14 days. In an embodiment, the method further comprises culturing the mammalian cell at a first temperature for about 2 days followed by culturing the mammalian cell at a second temperature for about 9, about 10, about 11, about 12, about 13, or about 14 days. In an embodiment, the method further comprises culturing the mammalian cell at a first temperature for about 3 days followed by culturing the mammalian cell at a second temperature for about 9, about 10, about 11, about 12, about 13, or about 14 days. In an embodiment, the method further comprises culturing the mammalian cell at a first temperature for about 4 days followed by culturing the mammalian cell at a second temperature for about 9, about 10, about 11, about 12, about 13, or about 14 days. In an embodiment, the method further comprises culturing the mammalian cell at a first temperature for about 5 days followed by culturing the mammalian cell at a second temperature for about 9, about 10, about 11, about 12, about 13, or about 14 days.

In an embodiment, the second temperature is lower than the first temperature. In an embodiment, the second temperature is about 1° C., about 2° C., about 3° C., about 4° C., about 5° C., or about 6° C. lower than the first temperature.

In an embodiment, the first temperature is 34° C.-40° C. In an embodiment, the first temperature is about 34° C., about 35° C., about 36° C., about 37° C., about 38° C., about 39° C., or about 40° C. In an embodiment, the first temperature is about 36° C.

In an embodiment, the second temperature is 30° C.-36° C. In an embodiment, the second temperature is about 30° C., about 31° C., about 32° C., about 33° C., about 34° C., about 35° C., or about 36° C. In an embodiment, the second temperature is about 33° C.

In an embodiment, the mammalian cell is cultured at the first temperature for 3-4 days. In an embodiment, the mammalian cell is cultured at the first temperature for about 3 days. In an embodiment, the mammalian cell is cultured at the second temperature for 11-12 days. In an embodiment, the mammalian cell is cultured at the second temperature for about 11 days.

In an embodiment, the mammalian cell is cultured in a bioreactor. In an embodiment, the mammalian cell is cultured in fed batch mode.

Examples of bioreactors include, but are not limited to, a plug flow bioreactor, a continuous stirred tank bioreactor, a fixed-bed bioreactor, an air-lift bioreactor, and a bioreactor bag.

Methods of adjusting the pH in a bioreactor are generally known in the art. In an embodiment, the method further comprises adding a base source to the cell culture medium if the pH goes below the deadband or adding an acid source to the cell culture medium if the pH goes above the deadband during the first period of time. In an embodiment, the method further comprises adding a base source to the cell culture medium if the pH goes below the deadband or adding an acid source to the cell culture medium if the pH goes above the deadband during the second period of time. In an embodiment, the acid source is CO₂. In an embodiment, the base source is a solution comprising NaOH.

In an embodiment, the mammalian cell culture medium is sparged with air during the first period of time. In an embodiment, the mammalian cell culture medium is sparged with air during the second period of time. In an embodiment, the mammalian cell culture medium is sparged with air during the first and second period of time. In an embodiment, the mammalian cell culture medium is sparged with air during the first and/or second period of time to reduce the pCO₂ in the cell culture medium. In an embodiment, the mammalian cell culture medium is sparged with air to raise the pH.

In an embodiment, the mammalian cell is selected from the group consisting of a COS cell, a CHO cell, a BHK cell, an MDCK cell, a HEK293 cell, a HEK293T cell, a HeLa cell, an NSO cell, a PER.C6 cell, a VERO cell, a CRL7030 cell, an HsS78Bst cell, an NIH 3T3 cell, a HepG2 cell, an SP210 cell, an R1.1 cell, a B-W cell, an L-M cell, a BSC1 cell, a BSC40 cell, a YB/20 cell, and a BMT10 cell. In an embodiment, the mammalian cell is a CHO cell.

The cell culture medium used in the methods disclosed herein can comprise any medium known to the skilled artisan that is suitable for culturing mammalian cells.

In an embodiment, one or more protease has reduced activity within the second pH deadband compared to the first pH deadband. In an embodiment, one or more protease has reduced activity at the second pH setpoint compared to the first pH setpoint. In an embodiment, the one or more protease is in the cell or the cell culture medium. In an embodiment, the one or more protease clips dulaglutide at the N-terminus, optionally wherein the protease is cathepsin D.

In an embodiment, less than about 4% of the dulaglutide produced is N-terminally clipped. In an embodiment, less than about 4%, about 3.9%, about 3.8%, about 3.7%, about 3.6%, about 3.5%, about 3.4%, about 3.3%, about 3.2%, about 3.1%, about 3%, about 2.9%, about 2.8%, about 2.7%, about 2.6%, about 2.5% of the dulaglutide produced is N-terminally clipped. In an embodiment, the N-terminally clipped dulaglutide does not have the N-terminal H1 or G2 residues of dulaglutide.

In an embodiment, less than about 4% of the Fc-containing protein produced is N-terminally clipped. In an embodiment, less than about 4%, about 3.9%, about 3.8%, about 3.7%, about 3.6%, about 3.5%, about 3.4%, about 3.3%, about 3.2%, about 3.1%, about 3%, about 2.9%, about 2.8%, about 2.7%, about 2.6%, about 2.5%, about 2.4%, about 2.3%, about 2.2%, about 2.1%, about 2.0%, about 1.9%, about 1.8%, about 1.7%, about 1.6%, about 1.5%, about 1.4%, about 1.3%, about 1.2%, about 1.1%, or about 1.0% of the Fc-containing protein produced is N-terminally clipped.

### III. Fc-Containing Proteins

The methods provided by the present disclosure are for the production of an Fc-containing protein by culturing a mammalian cell that expresses the Fc-containing protein.

In an embodiment, the Fc-containing protein comprises one or more of the amino acid sequences set forth in Table 1 below.

In an embodiment, the Fc-containing protein comprises a glucagon-like peptide 1 (GLP-1) analog comprising one or more modifications compared to a wild type GLP-1 amino acid sequence (SEQ ID NO: 1).

In an embodiment, the Fc-containing protein comprises a GLP-1 analog comprising the amino acid sequence of SEQ ID NO: 2.

In an embodiment, the Fc-containing protein comprises a peptide linker. In an embodiment, the C-terminal amino acid of the GLP-1 analog portion of the Fc-containing protein is fused to the N-terminus of an Fc portion of an immunoglobulin via a peptide linker. In an embodiment, the peptide linker comprises 1 to 10 G₄S units (SEQ ID NO: 3).

In an embodiment, the Fc-containing protein comprises: a GLP-1 analog comprising the amino acid sequence of SEQ ID NO: 2; a peptide linker comprising 1 to 10 G₄S units (SEQ ID NO: 3); and an Fc portion of an immunoglobulin. In an embodiment, the N-terminal residue of the peptide linker is directly fused to the C-terminal residue of the GLP-1 analog, and the C-terminal residue of the peptide linker is directly fused to the N-terminal residue of the Fc portion.

In an embodiment, the Fc-containing protein comprises the amino acid sequence of SEQ ID NO: 4. In an embodiment, the wherein the Fc-containing protein is a homodimer comprising two identical amino acid chains each comprising the amino acid sequence of SEQ ID NO: 4.

In an embodiment, the Fc-containing protein is dulaglutide.

Dulaglutide is a human GLP-1 receptor agonist which comprises a dimer of a GLP-1 analog fused at its C-terminus via a (G₄S)₃ peptide linker to the N-terminus of an analog of an Fc portion of an immunoglobulin, and is identified by CAS registry number 923950-08-7, which provides the following chemical name: 7-37-Glucagon-like peptide I [8-glycine, 22-glutamic acid, 36-glycine] (synthetic human) fusion protein with peptide (synthetic 16-amino acid linker) fusion protein with immunoglobulin G4 (synthetic human Fc fragment), dimer. Each monomer of dulaglutide has the amino acid sequence set forth in SEQ ID NO: 4.

The two monomers are attached by disulfide bonds between the cysteine residues at positions 55 and 58 of SEQ ID NO: 4 to form the dimer. Dulaglutide's structure, function, production, and use in treating T2DM is described in more detail in U.S. Pat. No. 7,452,966 and U.S. Patent Application Publication No. US20100196405. Dulaglutide agonizes the GLP-1 receptor resulting in stimulation of insulin synthesis and secretion and has been shown to provide improved glycemic control in T2DM patients.

When used herein, the term “dulaglutide” refers to any GLP-1 receptor agonist protein dimer of two monomers having the amino acid sequence of SEQ ID NO: 4, including any protein that is the subject of a regulatory submission seeking approval of a GLP-1 receptor agonist product which relies in whole or part upon data submitted to a regulatory agency by Eli Lilly and Company relating to dulaglutide, regardless of whether the party seeking approval of said protein actually identifies the protein as dulaglutide or uses some other term.

TABLE 1
Sequences comprised in certain Fc-containing
proteins

| | SEQ | |
|---|---|---|
| | ID | |
| Description | NO: | Amino acid sequence |
| WT GLP 1 | 1 | HAEGTFTSDVSSYLEGQAAKEFIAWLVKGRG |
| GLP 1 | 2 | HGEGTFTSDVSSYLEEQAAKEFIAWLVKGGG |
| analog | | |
| G4S | 3 | GGGGS |
| Dulaglutide | 4 | HGEGTFTSDVSSYLEEQAAKEFIAWLVKGGGGG |
| monomer | | GGSGGGGSGGGGSAESKYGPPCPPCPAPEAAGG |
| | | PSVFLFPPKPKDTLMISRTPEVTCVVVDVSQED |
| | | PEVQFNWYVDGVEVHNAKTKPREEQFNSTYRVV |
| | | SVLTVLHQDWLNGKEYKCKVSNKGLPSSIEKTI |
| | | SKAKGQPREPQVYTLPPSQEEMTKNQVSLTCLV |
| | | KGFYPSDIAVEWESNGQPENNYKTTPPVLDSDG |
| | | SFFLYSRLTVDKSRWQEGNVFSCSVMHEALHNH |
| | | YTQKSLSLSLG |

In an embodiment, the Fc-containing protein is etanercept, alefacept, abatacept, rilonacept, romiplostim, belatacept, aflibercept, conbercept, efmoroctocog alpha, eftrenonacog alpha, asfotase alpha, or luspatercept.

In an embodiment, the Fc-containing protein is an antibody. In an embodiment, the Fc-containing protein is not an antibody.

In an aspect, provided herein is an Fc-containing protein produced by any one of the methods disclosed herein.

In an aspect, provided herein is dulaglutide produced by any one of the methods disclosed herein.

### EXAMPLES

The following examples are offered by way of illustration, and not by way of limitation.

### Example 1: Analyzing Elevated pCO₂ Levels and a pH Setpoint Shift in a Production Bioreactor

This example describes a study that evaluated changes in the initial pH setpoint and deadband as well as implementing a shift in the pH setpoint and deadband during the production bioreactor stage of dulaglutide manufacturing, to identify parameters that reduced pCO₂ levels and minimized the level of clipped forms of dulaglutide (e.g., the N-terminal clipped species of dulaglutide, des H/HG, which is a variant of dulaglutide where the H1 residue or both H1 and G2 residues are clipped from the N-terminus of dulaglutide) in the product. In a dulaglutide manufacturing scheme, the initial pH setpoint and deadband were 6.80±0.03 in the production bioreactor stage. However, under these conditions, increased pCO₂ in the cell culture led to an increase in clipped forms of dulaglutide and thus, decreased dulaglutide titer.

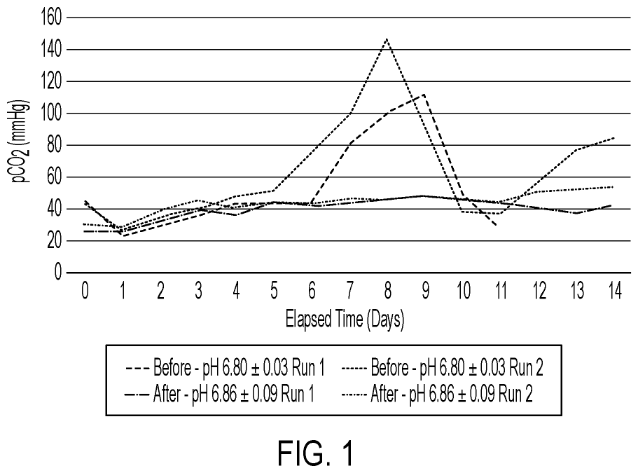

An initial study, assessing a change in the initial pH setpoint and deadband to 6.86±0.09, demonstrated that changing the initial pH setpoint and widening the deadband reduced the elevated pCO₂ observed with previous culture conditions in the production bioreactor stage (FIG. 1). Thus, under the conditions tested, an initial pH of 6.86±0.09 maintained lower levels of pCO₂. The small-scale studies described below investigate whether adding a pH shift could further optimize the cell culture conditions of this stage of dulaglutide production.

These small-scale studies were performed with equipment, the current dulaglutide working cell bank (WCB), raw materials, process schedules, and process conditions that could best mimic a dulaglutide commercial manufacturing cell culture process when performed at the laboratory scale. The test conditions were focused specifically on pH control during the production bioreactor stage of dulaglutide manufacturing.

The purpose of the following study was to implement CO₂ clamp software in order to mimic elevated pCO₂ levels and to evaluate the impact of a pH shift on day 11 on des H/HG levels. The conditions for this study are described in Table 2 below.

TABLE 2
Culture Conditions

| | | pH setpoints and | CO₂ Clamp |
|---|---|---|---|
| Condition | Replicates | deadbands | Used? |
| 1 | 5 | 6.86 ± 0.09 Days 0-14 | No |
| 2 | 8 | 6.86 ± 0.09 Days 0-14 | Yes |
| 3 | 3 | 6.86 ± 0.09 Days 0-11 | No |
| | | 7.00 ± 0.05 Days 11-14 | |
| 4 | 3 | 6.86 ± 0.09 Days 0-11 | No |
| | | 6.75 ± 0.02 Days 11-14 | |

The CO₂ clamp software in the Applikon 3 L bioreactor system used in these studies worked via an internal computer script that calculates the CO₂ gas flow based off a percentage of total gas flow (air sparge+O₂ sparge) and the manual input of specific pCO₂ target increase set points. The CO₂ clamp automatically shut off when the process was outside the pH deadband, thereby allowing the pH control (either CO₂ sparge cascade or 2N NaOH) to maintain the pH. For example, if the baseline pCO₂ without any additional CO₂ sparge is 30 mmHg and the CO₂ clamp software was set to 5 mmHg, the pCO₂ was expected to increase to 35 mmHg.

The viable cell densities (VCD) over the course of the 14 day culture in the four different conditions were comparable for all conditions tested. The viability of the cells was comparable in each condition until the end of the culture, when CO₂ clamp conditions resulted in slightly reduced cell viability. The concentrations of glucose in the cell culture media were comparable in each condition through the 14 day culture, with the high pH shift to 7.00±0.05 on day 11 requiring a glucose feed on day 13 to prevent glucose depletion prior to harvest on day 14. There were slight increases in the concentrations of lactate in the cell culture media observed in the conditions with the CO₂ clamp and pH shift to 6.75±0.02 on day 11 towards the end of the 14 day culture, otherwise, the concentrations of lactate in the media were comparable.

The sodium levels in the cultures were comparable for all conditions throughout the 14 days and the offline pH profiles are highly comparable until the day 11 pH shift. The pCO₂ in the CO₂ clamp condition (n=8) gradually increased after day 6 until about day 10 when the average pCO₂ leveled off at around 70 mmHg until the end of the run. Dulaglutide titers were comparable, except that the CO₂ clamp conditions resulted in a lower average day 14 titer.

In the four conditions tested in this study, the CO₂ clamp conditions resulted in the lowest average titer and greatest variability overall, which is likely due to the lactate concentrations observed in this set of bioreactor conditions, i.e., increased lactate resulted in decreased titer (Table 3). The day 11 pH setpoint shift to 7.00 had the highest average titer.

TABLE 3
Dulaglutide Titer results

| | Mean | Std | | |
|---|---|---|---|---|
| Condition | (mg/mL) | Dev | Min | Max |
| 1 (6.86 ± 0.09) | 2.186 | 0.175 | 1.88 | 2.319 |
| 2 (6.86 ± 0.09 and CO₂ clamp) | 1.988 | 0.233 | 1.608 | 2.291 |
| 3 (D11 shift to 6.75 ± 0.02) | 2.119 | 0.093 | 2.021 | 2.206 |
| 4 (D11 shift to 7.0 ± 0.05) | 2.24 | 0.108 | 2.118 | 2.323 |

The CO₂ clamp conditions resulted in a mean des H/HG value of 3.36% with several data points over the acceptance criteria of not more than 3.5%. Further, the day 11 pH setpoint shift to 6.75 (condition 4) resulted in a mean des H/HG value of 3.20% with two datapoints either near or above the acceptance criteria, while the day 11 pH setpoint shift to 7.00 (condition 3) resulted in the lowest average des H/HG of 2.27% (Table 4).

TABLE 4
Des H/HG results

| | Mean | Std | | |
|---|---|---|---|---|
| Condition | (mg/mL) | Dev | Min | Max |
| 1 (6.86 ± 0.09) | 2.56 | 0.482701 | 2.2 | 3.3 |
| 2 (6.86 ± 0.09 and CO₂ clamp) | 3.3625 | 0.434042 | 2.8 | 3.9 |
| 3 (D11 shift to 6.75 ± 0.02) | 3.2 | 0.556776 | 2.6 | 3.7 |
| 4 (D11 shift to 7.0 ± 0.05) | 2.26667 | 0.288675 | 2.1 | 2.6 |

A positive correlation was observed between the day 14 pCO₂ and des H/HG. A negative correlation was observed between the day 14 offline pH and des H/HG.

The results of this study demonstrated that elevated pCO₂ or a day 11 shift to a lower pH set point correlated with higher levels of des H/HG and lower dulaglutide titer, and a shift to a higher pH setpoint at day 11 correlated with lower levels of des H/HG and a higher dulaglutide titer.

### Example 2: Identifying Optimal pH Conditions in a Production Bioreactor

This study was designed to identify the optimal pH strategy for the production bioreactor stage of a dulaglutide manufacturing scheme, including pH shift day, pH setpoint and deadband shift magnitude, and pCO₂ levels for process robustness and critical quality attributes of the product, specifically levels of a clipped form of dulaglutide (des H/HG). The conditions used in this study are described in Table 5 below. All conditions were performed over a total of 14 days in a 5 L production bioreactor.

The CO₂ control in the Sartorius 5 L system used in these studies was controlled manually by performing daily calculations using total gas flow (air sparge+O₂ sparge) to

$$CO 2 flow ( ccm ) ) = \frac{Target CO 2 ( mmHg ) / 760 ) * ( Total O 2 flow ( ccm ) + Air flow ( ccm ) )}{( 1 - ( Target CO 2 ( mmHg ) / 760 ) )}$$

TABLE 5
Culture Conditions

| | pH Shift | pH Shift | CO₂ Target |
|---|---|---|---|
| Condition | Day | Magnitude | (mmHg) |
| Center Point (n = 2) | 9 | 0.14 (7.00 ± 0.05) | 70 |
| 1 | 8 | 0.07 (6.93 ± 0.05) | 60 |
| 2 | | | 80 |
| 3 | | 0.21 (7.07 ± 0.05) | 60 |
| 4 | | | 80 |
| 5 | 10 | 0.07 (6.93 ± 0.05) | 60 |
| 6 | | | 80 |
| 7 | | 0.21 (7.07 ± 0.05) | 60 |
| 8 | | | 80 |

Viable cell densities and viability levels were comparable for all conditions throughout the 14 day culture with slight decreases in viability in the last two days in conditions where an increase in lactate was observed. Glucose concentrations in the cell culture media were comparable until the pH shifts on days 8, 9, and 10, and a general increase in glucose consumption was observed in cultures with a pH shift to 7.07±0.05. An increase in lactate concentration in the media towards the end of the 14 days was observed in all tanks with high pCO₂.

The results from this study showed that the offline pH profiles and sodium levels were comparable until days 8, 9, and 10 when the different pH shifts were implemented. The dulaglutide titers were comparable on day 8 but were less so by day 14. The lowest titers were observed in the bioreactors where high pCO₂ and elevated lactate were also observed.

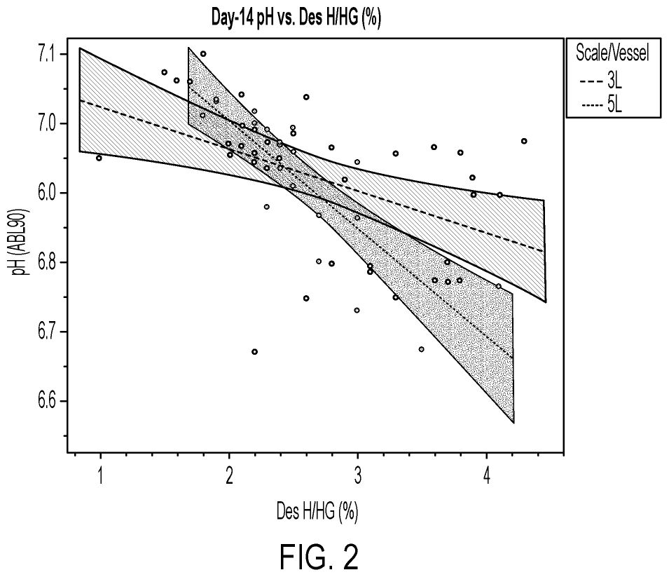

Analysis of the levels of des H/HG showed that the pH level on day 14 of the production bioreactor had a negative correlation with des H/HG levels in the production bioreactor studies (FIG. 2), suggesting that the pH shift to a higher pH setpoint and narrower deadband, to maintain a higher pH toward the end of the 14 days, was an optimal pH control strategy for the production bioreactor stage of dulaglutide production in this study.

Further analysis of the results from this study demonstrated that there was a statistically significant negative correlation between the post pH shift setpoint and the levels of des H/HG (a scaled correlation estimate of −0.25, p=0.0363), which indicated that a shift to a higher pH setpoint reduced the amount of protease clipping in culture. Further, there was a statistically significant negative correlation between pCO₂ and dulaglutide harvest titer (a scaled correlation estimate of −0.154, p=0.0077), which indicated that increases in pCO₂ could lead to a lower dulaglutide titer in the production bioreactor culture. The results also demonstrated that the pH shift day (day 8, 9, or 10) had minimal impact on or correlation with dulaglutide titer or des H/HG levels.

Taken together, the foregoing studies described in Examples 1 and 2 demonstrated that an initial pH setpoint and deadband of 6.86±0.09 mitigated the pCO₂ increases observed at a pH setpoint and deadband of 6.80±0.03. Further, shifting to a higher pH setpoint and narrower deadband at day 8, 9, 10, or 11 of cell culture reduced the level of des H/HG and increased dulaglutide titer, when compared to a cell culture that maintained the initial pH setpoint and deadband throughout cell culture.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

Other embodiments are within the following claims.

1. A method for producing dulaglutide, the method comprising the steps of:
a) culturing a mammalian cell that expresses the dulaglutide in a cell culture medium at a first pH setpoint for a first period of time; followed by
b) culturing the mammalian cell in the cell culture medium at a second pH setpoint for a second period of time, wherein the second pH setpoint is higher than the first pH setpoint,
such that the dulaglutide is produced by the mammalian cell.

2. The method of claim 1, wherein the first pH setpoint has a deadband of 0.01-0.10.

3. The method of claim 1, wherein the first pH setpoint has a deadband of 0.07-0.10.

4. The method of claim 1, wherein the first pH setpoint has a deadband of about 0.09.

5. The method of claim 1, wherein the second pH setpoint has a deadband of 0.01-0.10.

6. The method of claim 1, wherein the second pH setpoint has a deadband of 0.03-0.06.

7. The method of claim 1, wherein the second pH setpoint has a deadband of about 0.05.

8. The method of claim 1, wherein the deadband of the second pH setpoint is narrower than the deadband of the first pH setpoint.

9. The method of claim 1, wherein the first pH setpoint has a deadband of about 0.09 and the second pH setpoint has a deadband of about 0.05.

10. The method of claim 1, wherein the second pH setpoint is 0.01-1.0 pH units higher than the first pH setpoint.

11. The method of claim 1, wherein the first pH setpoint is 6.0-7.0.

12. The method of claim 1, wherein the first pH setpoint is 6.5-6.9.

13. The method of claim 1, wherein the first pH setpoint is 6.8-6.9.

14. The method of claim 1, wherein the first pH setpoint is about 6.86.

15. The method of claim 1, wherein the second pH setpoint is 6.5-7.5.

16. The method of claim 1, wherein the second pH setpoint is 6.9-7.5.

17. The method of claim 1, wherein the second pH setpoint is 7.0-7.1.

18. The method of claim 1, wherein the second pH setpoint is about 7.0.

19. The method of claim 1, wherein the first pH setpoint is about 6.86 with a deadband of about 0.09 and the second pH setpoint is about 7.0 with a deadband of about 0.05.

20. The method of claim 1, wherein the first period of time is longer than the second period of time.

21. The method of claim 1, wherein the first period of time is 7-12 days.

22. The method of claim 1, wherein the first period of time is 9-11 days.

23. The method of claim 1, wherein the first period of time is about 10 days.

24. The method of claim 1, wherein the second period of time is at least 1 day.

25. The method of claim 1, wherein the second period of time is 4-7 days.

26. The method of claim 1, wherein the second period of time is 4-6 days.

27. The method of claim 1, wherein the second period of time is about 5 days.

28. The method of claim 1, wherein the first period of time is about 10 days and the second period of time is about 5 days.

29. A method for producing dulaglutide, the method comprising the steps of:
a) culturing a mammalian cell that expresses the dulaglutide in a cell culture medium at a pH setpoint of about 6.86 with a deadband of about 0.09 for a first period of time comprising 9-10 days; followed by
b) culturing the mammalian cell in the cell culture medium at a pH setpoint of about 7.0 with a deadband of about 0.05 for a second period of time comprising 4-5 days,
such that the dulaglutide is produced by the mammalian cell.

30. The method of claim 1, further comprising culturing the mammalian cell at a first temperature for the first 1-5 days of the first period of time followed by culturing the mammalian cell at a second temperature for 9-14 days.

31. The method of claim 30, wherein the second temperature is lower than the first temperature.

32. The method of claim 31, wherein the first temperature is 34° C.-40° C.

33. The method of claim 31, wherein the first temperature is about 36° C.

34. The method of claim 31, wherein the second temperature is 30° C.-36° C.

35. The method of claim 31, wherein the second temperature is about 33° C.

36. The method of claim 31, wherein the mammalian cell is cultured at the first temperature for 3-4 days.

37. The method of claim 31, wherein the mammalian cell is cultured at the first temperature for about 3 days.

38. The method of claim 31, wherein the mammalian cell is cultured at the second temperature for 11-12 days.

39. The method of claim 31, wherein the mammalian cell is cultured at the second temperature for about 11 days.

40. The method of claim 1, wherein the mammalian cell is cultured in a bioreactor.

41. The method of claim 40, wherein the mammalian cell is cultured in fed batch mode.

42. The method of claim 1, further comprising adding a base source to the cell culture medium if the pH goes below the deadband or adding an acid source to the cell culture medium if the pH goes above the deadband during the first period of time.

43. (canceled)

44. The method of claim 42, wherein the acid source is CO₂.

45. The method of claim 42, wherein the base source is a solution comprising NaOH.

46. The method of claim 1, wherein the cell culture medium is sparged with air during the first and/or second period of time.

47. The method of claim 1, wherein the mammalian cell is selected from the group consisting of a COS cell, a CHO cell, a BHK cell, an MDCK cell, a HEK293 cell, a HEK293T cell, a HeLa cell, an NSO cell, a PER.C6 cell, a VERO cell, a CRL7030 cell, an HsS78Bst cell, an NIH 3T3 cell, a HepG2 cell, an SP210 cell, an R1.1 cell, a B-W cell, an L-M cell, a BSC1 cell, a BSC40 cell, a YB/20 cell, and a BMT10 cell.

48. The method of claim 1, wherein the mammalian cell is a CHO cell.

49. The method of claim 1, wherein one or more protease has reduced activity within the second pH deadband compared to the first pH deadband.

50. The method of claim 1, wherein one or more protease has reduced activity at the second pH setpoint compared to the first pH setpoint.

51. The method of claim 50, wherein the one or more protease clips dulaglutide at the N-terminus, optionally wherein the protease is cathepsin D.

52. The method of claim 1, wherein less than about 4% of the dulaglutide produced is N-terminally clipped.

53. The method of claim 52, wherein the clipped form of dulaglutide does not have the HG residues at the N-terminus of dulaglutide.

54. A method for producing dulaglutide in a CHO cell, the method comprising the steps of:
a) culturing the CHO cell in a cell culture medium maintained at a first pH setpoint of about 6.68 having a deadband of about 0.09 for about 10 days; followed by
b) culturing the CHO cell in the cell culture medium maintained at a second pH setpoint of about 7.0 having a deadband of about 0.05 for about 5 days; and
wherein the culturing is initiated at a temperature of about 36° C. and then decreased to about 33° C. after about 3 days;
such that the dulaglutide is produced by the CHO cell.

55. The method of claim 54, wherein the CHO cell is cultured in a bioreactor in fed batch mode.

56. The method of either claim 54 wherein NaOH is added to the cell culture medium if the pH goes below the deadband of either the first or second pH setpoint, and CO₂ is added to the cell culture medium if the pH goes above the deadband of either the first or second pH setpoint.

57. The method of claim 54 wherein the cell culture medium is sparged with air one or more times.

58. The method of claim 54 wherein less than about 4% of the dulaglutide produced by the CHO cell is N-terminally clipped.

59. Dulaglutide produced by the method of claim 1.
